SEQUENCE LISTING (1 sequence)

SEQ ID NO: 1 - protein, 15 aa, Homo sapiens
TLLDRRAHGYGAHLE

(19) United States
(12) Patent Application Publication
(10) Pub. No.: US 2008/0241945 A1
(43) Pub. Date: Oct. 2, 2008

(54) Peptide for differentiating osteoarthritis from rheumatoid arthritis and non-disease conditions

(76) Inventors: Pilar Lorenzo, Lund (SE); Tore Saxne, Lund (SE); Dick Heinegard, Lund (SE)

Correspondence Address: LYNN E BARBER, P O BOX 16528, FORT WORTH, TX, 76162, US

(21) Appl. No.: 11/728,856

(22) Filed: Mar. 27, 2007

(51) Int. Cl. G01N 33/68; C07K 16/18; C07K 7/08

(52) U.S. Cl. 436/86; 530/326; 530/387.9

(57) ABSTRACT

A method for differentiation of osteoarthritis from rheumatoid arthritis and non-disease conditions in a sample, comprising measuring in the sample the concentration of a peptide comprising the 15 amino acid sequence (SEQ ID NO: 1) of the human cartilage intermediate layer protein 2.

### FIELD OF INVENTION

The present invention relates to a method for aiding in the assessment of osteoarthritis (OA). The method especially is used in assessing the absence or presence of OA. The method is for example practiced by analyzing biochemical markers, comprising measuring in a sample the concentration of a 15 amino acid peptide immunogen (SEQ ID NO :1) related to the N-terminal part of human cartilage intermediate layer protein 2 (CILP-2) in body fluids and correlating the concentrations determined to the absence or presence of OA. This invention also describes development of diagnostic and prognostic assays for differentiation of osteoarthritis (OA) from rheumatoid arthritis (RA) and non-disease conditions.

### BACKGROUND

Arthritis is a group of conditions that affect the health of the joints in the body, including rheumatoid arthritis and psoriatic arthritis, which are autoimmune diseases; septic arthritis, caused by joint infection; and the more common osteoarthritis. Unlike the autoimmune diseases, osteoarthritis largely affects older people and results from the degeneration of joint cartilage.

Osteoarthritis is the most common form of arthritis affecting a large part of the population. Although osteoarthritis can affect almost any joint, it most often affects the hands, knees, hips, and spine. Common symptoms include pain, stiffness, loss of joint motion, and changes in the shape of affected joints. It is frequently called degenerative joint disease or “wear and tear” arthritis. Although it can be brought on suddenly by an injury, its onset is generally gradual in which aging brings on a breakdown in cartilage, and pain gets progressively more severe, although in early stages it can be relieved with rest. Dull, throbbing nighttime pain is characteristic, and it may be accompanied by muscle weakness or deterioration. Symptoms usually appear after the age of 50 and progress slowly. Starting with joint pain, the condition progresses and eventually the joint becomes deformed, limiting movement. As the cartilage breaks down it leaves the bone exposed, altering the person's gait. Later stages of the disease have been shown to have a component of inflammation, where the process in the cartilage may have a role in stimulating this inflammation. The condition is believed to be initiated by excessive or unusual load on the joint, where overweight, poor posture, repetitive strain from work, injury, sports injury or a combination of these factors are known to increase the risk.

There are also components of new production of tissue structures, particularly evident in the form of the so-called osteophytes that are new structures formed by endochondral bone formation. Although mechanical factors appear to have a role in both disease initiation and progression, little is known about specific events, partly due to the lack of diagnostic procedures that can identify those early stages of the disease. Patients usually seek care due to pain and the joint malfunction late in disease development, when cartilage destruction has already advanced significantly.

Today there is no single sign, symptom, or test result that allows a definitive diagnosis of osteoarthritis. Instead, the diagnosis is based on consideration of several factors, including presence of the characteristic signs and symptoms of osteoarthritis and the results of laboratory tests and x-rays, criteria set by The American College of Rheumatology (ACR).

Radiographs can usually confirm the diagnosis of osteoarthritis, although the findings are nonspecific. The cardinal radiographic features of the disease are loss of joint space and presence of new bone formation or osteophytes. The association between joint pain and radiographic features of osteoarthritis is not very close, such that even joints with pathologic or radiographic evidence of this disease may remain asymptomatic. Another shortcoming of using radiography for depicting the level of cartilage destruction in OA, particularly for the knee, is the necessity to have the exact angle of the X-rays for a correct measure of the joint space. Diagnosis using X-rays is used several years after the onset of the injury, whereas in the invention, bio-markers, such as SEQ ID NO: 1 and fragments thereof can be used much earlier for proper diagnosis.

It is not clear what the underlying process in the progressive tissue destruction of osteoarthritis is, but there are clear events of breakdown of the major tissue macromolecules caused by increased proteolytic activity. It has been shown that the early event in this progressive tissue destruction is degradation of aggrecan (a proteoglycan that is a major structural component of cartilage), where five specific sites along the molecule can be cleaved by the so-called aggrecanases (ADAMTS-4 and 5). However, the normal levels of aggrecan are adapted to e.g. altered mechanical load of the cartilage in a process that involves cleavage of the molecule at the typical sites and by the same ADAMTS-enzymes. In this process, there is fragmentation of collagen accomplished by specific collagenases and other enzymes that will degrade major molecules like cartilage oligomeric matrix protein (COMP).

In the process of OA some of the fragments produced are no longer retained in the tissue and are released into the surrounding body fluids and may eventually reach the circulation. This invention is based on measuring such fragments in body fluids as an indicator of the active process leading to tissue destruction. This molecular marker technology offers possibilities for new diagnostic procedures. These have the potential to detect much earlier events in the tissue destruction than is possible with the currently used approaches. It has been observed that when increased levels of circulating COMP fragments that have been released into synovial fluid eventually reach the blood, they can be used as a prognostic indicator of the process that will lead to destruction of the articular cartilage as observed by x-ray imaging. Although the processes in the diseases of osteoarthritis and rheumatoid arthritis are different, it has been found that the serum COMP levels have a prognostic value in both cases.

One limitation in evaluating the significance of altered COMP-levels in body fluids is the difficulty in distinguishing whether the majority of the COMP detected originates from normal turnover or disease progression. Other indicators that have been utilized include the C-terminal telo-peptide released upon cleavage of collagen type II, (referred to as CTX-II). Other assays directly measure new ends within the original polypeptide chain that are formed when collagen type II is cleaved by collagenases. An assay directed at the repair phase makes use of release of the C-terminal propeptide of collagen type II (CP-II) when procollagen is processed for collagen fibrillogenesis. This propeptide is apparently not retained in the cartilage. Procedures to measure release of aggrecan fragments have limited use, since the major fragments containing the negatively charged chondroitin sulfate chains appear to be largely eliminated in the lymph nodes without reaching the circulation (Frazer, Heinegård, Saxne, unpublished data). However, measurements of aggŕecan fragments in synovial fluid from patients with early rheumatoid arthritis have proven to identify those patients that develop more extensive cartilage destruction over a 10 year period (1).

One obvious shortcoming of all these markers is the lack of specificity for a given joint disease and overlap of measured levels between samples from normal individuals and those with joint disease. Furthermore there is no or little distinction observed between cases with rheumatoid arthritis and osteoarthritis with any of these indicators. Only a portion of patients show values sufficiently elevated to clearly distinguish them from normal individuals (2).

One issue is that there is a continuous turnover of tissue structural molecules in response to regular and frequent load. This serves to adapt tissue function to new requirements, including removing fatigued tissue elements. One consequence of this turnover is that there is a continuous release of fragments generated by these normal cleavages. In current assays of fragments as molecular indicators there is little distinction between those generated by normal turnover and those generated by a pathological process. Thus there is a high background, which hampers the ability to detect an increased pathological molecular process. It is however possible that some of the collagen type II (collagen, abundant in articular cartilage) breakdown products may distinguish more clearly between normal and pathological events, even if the process may be induced by the same enzyme. This is possible since normal collagen turnover as shown for articular cartilage is orders of magnitude slower than for other matrix constituents.

As used herein, the proteins in which cartilage intermediate layer protein is the precursor are referred to as CILP-1 and CILP-2 respectively. The N-terminal part that we study is referred to as cartilage intermediate layer protein 2 C1, which is distinct from cartilage intermediate layer protein 2 C2.

C1 and CILP-2C1 (2C1) respectively and the NTPPHase part CILP-1C2 and CILP-2C2 respectively where the C denotes “chain”.

Cartilage intermediate layer protein (CILP), a large secreted glycoprotein (3-6) is thought to play a role in cartilage scaffolding (7) has also been claimed to have nucleoside triphosphate pyrophosphohydrolase [NTPPPH] activity (8-11). The expression of CILP appears to be largely restricted to cartilage (3,4,9,11,12). The amount of CILP protein increases in aging human articular cartilage, and CILP is one of only a few cartilage matrix proteins whose expression becomes markedly up-regulated in early osteoarthritis (4). In normal cultured porcine chondrocytes, transforming growth factor β1 (TGFβ1) induces CILP expression, whereas insulin-like growth factor 1 (IGF-1) suppresses CILP expression (10). The originally detected CILP is now referred to as cartilage intermediate layer protein 1 C1 (UniProtKB/Swiss-Prot entry 075339)

In the nucleotide sequence of a protein, cartilage intermediate layer protein 2 (CILP-2 was deposited in the Genbank sequence databank (Accession AF542080, year 2002). The first study of the protein CILP-2 appeared in 2003 (13), when it was found that it did not show nucleotide pyrophosphatase phosphodiesterase(NPP)activity (13).

CILP-2 has a 50% homology to CILP-1 and data (Lorenzo and Heinegård, unpublished) indicate that it is similarly cleaved into corresponding cartilage intermediate layer protein 2 C1 and cartilage intermediate layer protein 2 C2. (UniProtKB/Swiss-Prot entry Q8IUL8). From proteomics approaches both proteins are found in cartilage extracts (Önnerfjord and Heinegård, unpublished).

In recent work we have shown upregulation in both early and late stages of osteoarthritis of production of COMP, fibronectin and at the same time a new protein that we characterized and named CILP, now cartilage intermediate layer protein 1 C1 (3,4,16).

The peptide sequence Thr-Leu-Leu-Asp-Arg-Arg-Ala-His-Gly-Tyr-Gly-Ala-His-Leu-Glu (SEQ ID NO: 1) of the human cartilage intermediate layer protein 2 C1 has now surprisingly been shown by the inventors herein to be a marker which can be used to for differentiation of osteoarthritis from rheumatoid arthritis and non-disease conditions.

Studies implicate CILP (cartilage intermediate layer protein 1 C1) as an autoantigen in patients with osteoarthritis (14,15). There are no studies presented to indicate that cartilage intermediate layer protein 2 C1 may be altered in osteoarthritis. No article or patent could be found to show or suggest that cartilage intermediate layer protein 2 C1 or fragments thereof, specifically (SEQ1)may be used in the diagnosis of osteoarthritis.

Work by Du et al 2005 (14) implicated that a small proportion of patients with knee osteoarthritis had auto antibodies to CILP (cartilage intermediate layer protein 1 C1) ly-stage of knee osteoarthritis. Antibodies were only detected in 25/136 of the OA patients. Similarly Tsuruha et al. 2001 (7) detected only 8-10.5% antibodies to different regions of CILP (cartilage intermediate layer protein 1 C1). No studies have been reported to indicate antibodies to cartilage intermediate layer protein 2 C1 or fragments thereof.

In U.S. Pat. No. 6,124,095 and U.S. Pat. No. 6,251,389 assigned to Incyte, CILP-2 and polynucleotide encoding CILP-2 are disclosed. In these patents, the protein is denominated human nucleotide pyrophosphohydrolase-2 (NTPPH-2), but the NTPPH-2 sequence is identical to CILP-2. They noted the expression of NTPPH-2 in rheumatoid and osteoarthritic synovial capsule. Unlike the invention herein this patent do not describe the possibility of using NTPPH-2 for selective identification of OA patients. The same applicant has a granted patent (U.S. Pat. No. 5,876,963) on CILP-1 (NTPPH-1) and polynucleotide encoding NTPPH-1.

DE 10328033 (S. Blaess) describes chip carrying DNA sequences associated with osteoarthritis and rheumatoid arthritis e.g. for diagnosis, monitoring and drug development. This patent does not mention cartilage intermediate layer protein 2 C1.

WO03/054166 (Incyte) describes methods for determining susceptibility of an individual, preferably an OA patient, to joint space narrowing and/or osteophyte development and/or joint pain comprising identifying whether the individual has at least one polymorphism in a polynucleotide encoding a protein, one of many proteins mentioned is CILP. However the do not mention cartilage intermediate layer protein 2 C1.

WO02/095415 and WO01/38872 (Osteometer Biotech) both describe an assay for the diagnosis of the severity of osteoarthritis or rheumatoid arthritis comprising detecting an isomerized or optically inverted protein or fragment of a protein in a sample. Neither the fragment described in WO02/095415 or protein WO01/38872 is from cartilage intermediate layer protein 2 C1.

WO00/20018 (Univ. of California) describes a method for identifying a risk for an arthritic disorder, e.g. osteoarthritis, comprising comparing the level of at least one indicator, e.g. NTPPH, of altered mitochondrial function in a biological sample with a control sample.

Proper diagnosis of osteoarthritis is currently possible only at advanced disease and depends on X-ray and clinical investigations. In the case of rheumatoid arthritis destruction of the joint cartilage can only be determined at advanced stages by X-ray.

RA can worsen very quickly in its early stages and serious damage to the joints may occur in as short a time span as 24 months. When modern, effective treatments for RA, such as blocking TNF-α activity are initiated early, symptoms can be relieved and the worsening of joint destruction slowed, and early disability can be avoided.

There is no documented disease-modifying treatment of osteoarthritis. At present, no cure is available and treatment focuses on relieving pain. Common treatments include the use of non-steroidal anti-inflammatory drugs (NSAID's), which are often used to relieve pain Compounds such as chondroitin and glucosamine are thought to improve the cartilage itself, but well controlled studies remain an important focus.

In severe cases, joint replacement often becomes necessary. In a few cases joints may be fused. This procedure stops the pain, but results in permanent loss of joint function. Another treatment, not yet used for fully developed osteoarthritis, includes the transplantation of cultured autologous chondrocytes. If the condition persists without correction and/or therapy, the joint is destroyed, leading to major replacement surgery with total prosthesis, or to disability.

Thus, to introduce new therapeutic regimens that may stop the early stages of disease development new, early and correct diagnosis is of essence and would provide a breakthrough. For this reason the inventors of this application attempted to develop an assay that can be used as an indicator for developing osteoarthritis, as well as for differentiation of osteoarthritis from rheumatoid arthritis as well as a normal joint.

In early experiments the inventors could show that cartilage intermediate layer protein 1 C1, although upregulated in osteoarthritis, including both the early and late stages, did not show a marked increase in synovial fluid from patients with osteoarthritis and was not significantly different in fluid from patients with rheumatoid arthritis. Upon proceeding to develop an assay for cartilage intermediate layer protein 2 C1, we surprisingly showed that this protein acted as an indicator that showed unexpected and uniquely elevated levels in osteoarthritis. Levels in serum and synovial fluid are highly elevated and show no overlap with samples from rheumatoid arthritis and normal individuals. This is the first time that an assay for any protein released from a tissue has shown such a difference between samples representing different joint disease categories. The invention herein provides a novel diagnostic and prognostic assay for differentiation of osteoarthritis from rheumatoid arthritis as well as to monitor alterations in the activity of the disease destroying process during disease progression and following therapeutic intervention. Other objects and advantages will be more fully apparent from the following disclosure and appended claims.

### SUMMARY OF THE INVENTION

The invention provides a method for differentiation of osteoarthritis from rheumatoid arthritis and non-disease conditions in a sample, comprising measuring in the sample the concentration of a peptide comprising the 15 amino acid sequence (SEQ ID NO: 1) of the human cartilage intermediate layer protein 2 C1.

### BRIEF DESCRIPTION OF THE DRAWINGS

FIG. 1A is a graph showing samples of serum and FIG. 1B is a graph showing samples of knee joint synovial fluids from patients described in Example 4. The samples were analyzed by the ELISA for SEQ ID NO: 1 of the cartilage intermediate layer protein 2 C1.

### DETAILED DESCRIPTION OF THE INVENTION AND PREFERRED EMBODIMENTS THEREOF

The work with respect to the invention herein is based on early data on cartilage intermediate layer protein 1 C1, which was identified as one of a few proteins showing a major increase in osteoarthritis. Our first attempt with our antibody raised against the protein purified from the tissue was promising indicating that the protein was released into the synovial fluid from osteoarthritis patients and that the highest level was found in a sample from an osteoarthritis patient.

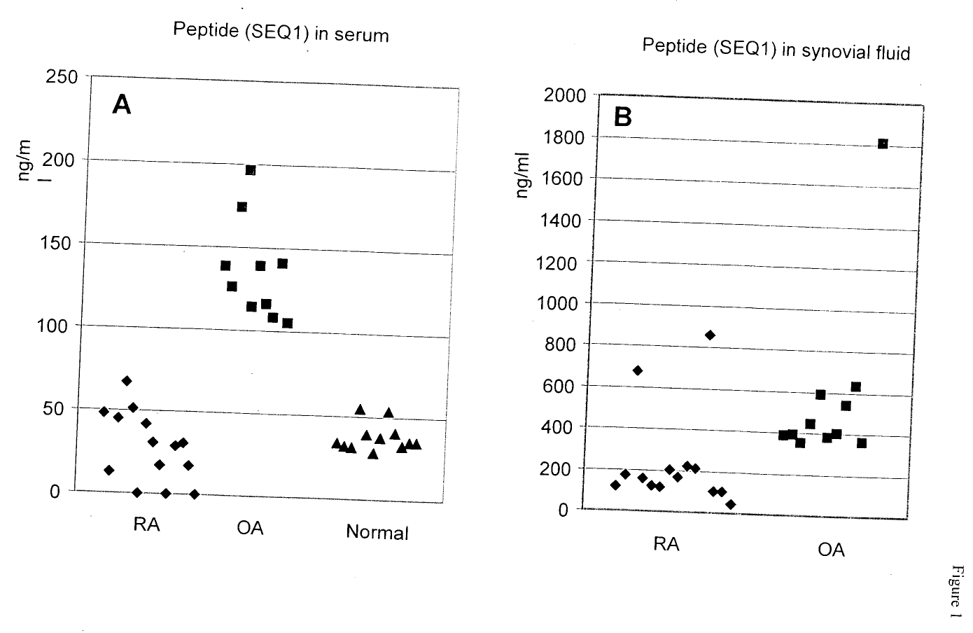

Work with the recombinant cartilage intermediate layer protein 1 C1 produced in EBNA 293 fibroblasts confusingly showed that this pure protein as the coating antigen in ELISA did not result in good inhibition levels with synovial fluid samples. At this time cartilage intermediate layer protein 2 appeared in the databases and we suspected that there was a contamination of antibodies to this protein in our preparation. We therefore developed a specific antibody to cartilage intermediate layer protein 2 C1, and have now used this to develop an assay for this protein intact or fragmented in synovial fluid and and blood. This assay turned out to be very promising and preliminary analyses of serum samples from normal individuals as well as patients with rheumatoid arthritis and osteoarthritis gave the results depicted in FIG. 1A and FIG. 1B.

The results showed that the levels of the peptide (SEQ ID NO: 1) were much higher in osteoarthritis compared to both rheumatoid arthritis and normal individuals, with no overlap. This is the first time that an assay has shown such a difference between samples representing different joint disease categories for any protein released from a tissue.

Serum and synovial fluid samples from fourteen patients with clinically established rheumatoid arthritis according to the ACR-criteria (all with knee joint arthritis), ten patients with clinically established knee joint osteoarthritis according to the clinical and radiographic ACR-criteria and twelve normal control serum samples from blood donors were analyzed with the established ELISA procedure. A central observation was that the levels of cartilage intermediate layer protein 2 C1 or fragments thereof, including SEQ ID NO: 1 were distinctly higher in the samples from patients with osteoarthritis, with no significant overlap to levels in the normal individuals, which in turn showed levels very similar to those in samples from patients with rheumatoid arthritis. The patients with osteoarthritis showed a wider range of considerably higher levels demonstrating that the release of increased levels of cartilage intermediate layer protein 2 C1 was a common denominator for this group.

The results show a unique difference in molecular marker levels between different conditions affecting the joint. Interestingly abundant data show that COMP levels in serum show elevated levels both in rheumatoid arthritis and osteoarthritis. Therefore ratios between COMP and cartilage intermediate layer protein 2 C1 distinguish individuals with rheumatoid arthritis from those normal, particularly in the subgroup of patients that appeared to show subnormal levels of cartilage intermediate layer protein 2 C1 reactivity.

The results unravel a novel molecular marker that has the potential to serve in the diagnosis of conditions with osteoarthritis. Levels of cartilage intermediate layer protein 2 C1 are distinctly higher than in normal individuals and in patients with rheumatoid arthritis. The difference between levels in normal individuals and those with osteoarthritis indicate that the assay of cartilage intermediate layer protein 2 C1 also serves as an indicator of existing disease activity. The wide range of values in patient samples indicates that the level correlate to the intensity of the process.

### EXAMPLE 1

Preparation of the Antigen and Antiserum

A synthetic peptide covering the amino acids 331-345 (SEQ ID NO: 1) of the human CILP-2 (GeneBank accession nr. Q8IUL8) was used as immunogen. An additional cysteine residue was added at the amino termini to allow selective coupling to different substrates. The peptide sequence (SEQ ID NO: 1) was used as immunogen after conjugation in its N-terminal via an added cysteine to keyhole limpet hemocyanin (KLH) for the production of polyclonal antibodies according to standard protocols.

A commercial source (Innovagen AB, Lund, Sweden) was used for the synthesis of the peptide, the conjugation to a carrier, the preparation of the antigen for immunization, including the injection to the rabbit and the production of the antiserum.

### EXAMPLE 2

Purification of the anti peptide antibody from the crude antiserum The generated antiserum was affinity purified on a column with the immobilized peptide (SEQ ID NO: 1) from cartilage intermediate layer protein 2 C1 (Innovagen AB, Lund, Sweden). The column (1.5 ml gel) was equilibrated with phosphate buffered saline (PBS, 0.1 M phosphate buffer, 150 mM NaCl, pH 7.5) and 5 ml of serum were applied and incubated end over end for 1 h at room temperature then further incubated for 1 h without mixing. The column was washed with 15 and then with 10 ml PBS containing 1 M NaCl. The column was eluted step wise with 1.5 ml of 100 mM Glycine pH 2.7. Ten fractions were collected and neutralized immediately with 50 μl of 1M Tris pH 9.5. Fractions with the highest absorbance were pooled and dialyzed against PBS containing 0.05% sodium azide. After dialysis the volume was measured and the concentration of the IgG was determined by its OD at 280 nm. The affinity purified antibody, stored frozen at −20° C. in 200 μl aliquots, was used in all the assays.

### EXAMPLE 3

Competitive Enzyme Linked Immunosorbent assay (ELISA) for SEQ ID NO. 1 of the Cartilage Intermediate Layer Protein 2 C1

A specific competitive ELISA was developed to measure human cartilage intermediate layer 2 C1 in body fluids.

1. Biotinylation of the peptides: Peptides were biotinylated via their terminal cysteine with EZ-Link® Maleimide PEO₂-Biotin as described by the manufacturer (PIERCE).

2. Pre-treatment of the antibody: The affinity purified peptide antibody was diluted 1:50 in phosphate buffered saline (PBS), pH 7.4 containing 5% n,n-dimethylformamide (Sigma-Aldrich). After incubation for 1 h at room temperature the antibody was diluted to 1:2000 with 4% Triton in 10 mM phosphate (NaH₂PO₄) pH 7.5.

3. Pre-treatment of the standard and samples: Standard (from 1 to 125 ng/ml) in 1% (w/v) sodium dodecyl benzene sulfonate (SDBS, Sigma-Aldrich) in 0.1 M sodium chloride, 0.05 M sodium phosphate pH 7.5 containing 0.5% bovine serum albumin (BSA, Sigma-Aldrich) and an appropriate dilution of synovial fluids or sera in 1% (w/v) SDBS solution without BSA were incubated overnight at room temperature. Samples that may be analyzed by the method of the invention include synovial fluid, blood, plasma, serum and urine.

4. Assay: 96-well microtiter plates (Nunc-Immunoplates, Maxisorp, Nunc Intermed Ltd, Copenhagen, Denmark) were coated overnight at room temperature in a wet chamber with 50 μl of streptavidin (ImmunoPure® Streptavidin, PIERCE) in PBS pH 7.4. After rinsing the plates with 0.15 M sodium chloride and 0.05% (w/v) Tween 20 the free binding sites of the polystyrene surface were blocked with 80 μl of 2 mg/ml bovine serum albumin (Sigma-Aldrich) in PBS, pH 7.4 for 1 h at room temperature. Then biotinylated peptide diluted 1:10000 was added and incubated for 1 h at room temperature. Thirty microliters of pre-treated standard (from 1 to 125 ng/ml) and samples of synovial fluids or sera (obtained by usual puncture) were mixed with 30 μl of diluted antibody. After 1 h preincubation at room temperature 50 μl of the mixture was added to the coated wells of the microtiter plate and further incubated for 1 h at room temperature. The plates were rinsed as above and the bound antibodies were detected by adding 50 μl of a dilution of rabbit anti-swine IgG conjugated with alkaline phosphatase (DAKO A/S, Denmark) in 0.1 M sodium chloride, 0.05 M sodium phosphate, 0.05% Tween 20, pH 7.5 containing 2 mg/ml of BSA. After 1 h incubation at room temperature the plates were rinsed as above and 50 μl of substrate was added (1 mg/ml p-nitrophenyl phosphate in 1M diethanolamine pH 9.8 containing 0.5 M MgCl₂).

The absorbance of each sample and standard was measured at 405 nm in duplicate by a microplate reader (Expert 96, AsysHitech, Austria). The Mikrowin 200 software program (AsysHitech, Austria) was used to plot the calibration curve and to calculate the content of CILP-2 in the samples analyzed.

### EXAMPLE 4

Study design

Fourteen patients with clinically established knee joint rheumatoid arthritis according to the ACR-criteria, ten patients with clinically established knee joint osteoarthritis according to the ACR-criteria and twelve normal control serum samples from blood donors were analyzed with the established ELISA procedure, FIG. 1.

### REFERENCES

1. Saxne T, Wollheim F, Pettersson H, Heinegård D. Brit. Proteoglycan concentration in synovial fluid: predictor of future cartilage destruction in rheumatoid arthritis. Med. J. (1987), 295, 1447-1448.)
2. Lindqvist E, Eberhardt K, Bendtzen K. Heinegård D, Saxne T. Ann Rheum Dis. Prognostic laboratory markers of joint damage in rheumatoid arthritis. (2005) 64, 196-201.
3. Lorenzo P, Neame P, Sommarin Y, Heinegård D. Cloning and deduced amino acid sequence of a novel cartilage protein (CILP) identifies a proform including a nucleotide pyrophosphohydrolase. J Biol Chem 1998;273:23469-75.
4. Lorenzo P, Bayliss MT, Heinegård D: A novel cartilage protein (CILP) present in the mid-zone of human articular cartilage increases with age. J Biol Chem 1998;273:23463-8.
5. Lorenzo P, Aman P, Sommarin Y, Heinegård D. The human CILP gene: exon/intron organization and chromosomal mapping. Matrix Biol 1999;18:445-54.
6. Nakamura I, Okawa A, Ikegawa S, Takaoka K, Nakamura Y. Genomic organization, mapping, and polymorphisms of the gene encoding human Cartilage intermediate layer protein. J Hum Genet 1999;44:203-5.
7. Tsuruha J, Masuko-Hongo K, Kato T, Sakata M, Nakamura H, Nishioka K. Implication of Cartilage intermediate layer protein in cartilage destruction in subsets of patients with osteoarthritis and rheumatoid arthritis. Arthritis Rheum 2001;44:838-45.
8. Masuda I, Hamada J-I, Haas A, Ryan L, McCarty D. A unique ectonucleotide pyrophosphohydrolase associated with porcine chondrocyte-derived vesicles. J Clin Invest 1995;95:699-704.
9. Masuda I, Halligan B D, Barbieri J T, Haas A L, Ryan L M, McCarty D J. Molecular cloning and expression of a porcine chondrocyte nucleotide pyrophosphohydrolase. Gene 1997; 197:277-87.
10. Hirose J, Masuda I, Ryan L M. Expression of Cartilage intermediate layer protein/nucleotide pyrophosphohydrolase parallels the production of extracellular inorganic pyrophosphate in response to growth factors and with aging. Arthritis Rheum 2000;43:2703-11.
11. Masuda I, lyama K-I, Halligan B D, Barbieri J T, Haas A L, McCarty D J, et al. Variations in site and levels of expression of chondrocyte nucleotide pyrophosphohydrolase with aging. J Bone Miner Res 2001;16:868-75.
12. Johnson K, Hashimoto S, Lotz M, Pritzker K, Goding J, Terkeltaub R. Up-regulated expression of the phosphodiesterase nucleotide pyrophosphatase family member PC-1 is a marker and pathogenic factor for knee meniscal cartilage matrix calcification. Arthritis Rheum 2001;44:1071-81.
13. Johnson K., Farley D., Hu S., Terkeltaub R. One of Two Chondrocyte-Expressed Isoforms of Cartilage Intermediate-Layer Protein Functions as an Insulin-Like Growth Factor 1 Antagonist. Arthritis Rheum Vol. 48, No. 5, May 2003, pp 1302-1314.
14. Du H., Masuko-Hongo K., Nakamura H., Xiang Y., Bao C-D., Wang X-D.,; Chen S-L., Nishioka K., Kato T. The prevalence of autoantibodies against Cartilage intermediate layer protein, YKL-39, osteopontin, and cyclic citrullinated peptide in patients with early-stage knee osteoarthritis: evidence of a variety of autoimmune processes. Rheuinatology international, (2005 November) Vol. 26, No. 1, pp. 35-41. Electronic Publication: 18 Sep. 09.
15. Kato Tomohiro; Xiang Yang; Nakamura Hiroshi; Nishioka Kusuki. Neoantigens in osteoarthritic cartilage. Current opinion in rheumatology, (2004 September) Vol. 16, No. 5, pp. 604-8.
16. Lorenzo P, Bayliss M, Heinegård D. Altered patterns and synthesis of extracellular matrix macromolecules in early osteoarthritis. Matrix Biol. (2004) 23, 381-91).

What is claimed Is:

1. A method to monitor joint disease by measuring in a sample fragments comprising a cartilage intermediate layer protein 2 C1 peptide sequence (SEQ ID NO: 1)

2. The method of claim 1, wherein the sample is selected from the group consisting of synovial fluid, blood, plasma, serum and urine.

3. A peptide for differentiating osteoarthritis from rheumatoid arthritis and non-disease conditions comprising SEQ ID NO: 1.

4. An antibody immunoreactive with a peptide comprising the amino acid sequence (SEQ ID NO: 1).

5. An assay to measure the peptide comprising the amino acid sequence (SEQ ID NO: 1), comprising analyzing a sample for the peptide using the antibody of claim 4.
